(19) United States
(12) Patent Application Publication
(10) Pub. No.: US 2003/0091553 A1
(43) Pub. Date: May 15, 2003

(54) Use of histamine to treat liver disease

(76) Inventor: Kurt R. Gehlsen, Encinitas, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/270,713

(22) Filed: Oct. 11, 2002

Related U.S. Application Data
(60) Provisional application No. 60/343,628, filed on Oct. 19, 2001.
(60) Provisional application No. 60/340,011, filed on Oct. 30, 2001.

(51) Int. Cl. A61K 38/44; A61K 31/355; A61K 31/375; A61K 31/07

(52) U.S. Cl. 424/94.4; 514/458; 514/474; 514/725

(57) ABSTRACT

The disclosure relates to methods for treating and/or preventing hepatic tissue and cell damage caused by reactive oxygen species in mammals. More specifically, the disclosure relates to the prevention and/or reduction of hepatic tissue and cell damage through the administration of histamine and histamine agonists.

### RELATED APPLICATIONS

This application claims priority to U.S. Provisional Application Ser. No. 60/343,628, filed on Oct. 19, 2001, and U.S. Provisional Application Ser. No. 60/340,011, filed on Oct. 30, 2001. The entire contents of these provisional applications are hereby incorporated by reference in their entireties.

### BACKGROUND OF THE INVENTION

1. Field of the Invention

The disclosure below relates to methods for treating and/or preventing hepatic tissue and cell damage caused by reactive oxygen species in mammals. More specifically, the disclosure relates to the prevention and/or reduction and/or reversal of hepatic tissue and cell damage through the administration of histamine and histamine-related compounds.

2. Description of the Related Art

Oxidative stress, i.e. toxicity inflicted by reactive oxygen species (ROS), is being recognized as a systemic phenomenon in liver disease, whose extent appears to correlate with the severity and stage of disease. The mechanism of action associated with the cellular damage caused by oxidative stress has been implicated in a number of diseases including hepatitis and relates to direct damage of hepatic cells. One author has examined a role for oxidative stress in the development of the hyperdynamic circulation in portal hypertension. Bomzon and Ljubuncic have indicated, however, that it is premature to conclude that oxidative stress per se impacts at least vascular smooth muscle cell function in liver disease. Pharmacol Ther., 89(3):295-308 (2001).

The theory that oxidative stress may play a role in liver disease may not be surprising as oxidative stress has been proposed to contribute to the state of immunosuppression at the site of malignant tumors and in chronic viral infections. (See U.S. Pat. Nos. 5,728,378, 6,000,516, and 6,155,266). Lymphocytes residing within or adjacent to tumors display signs of oxidative damage, including a higher degree of apoptosis and a defective transmembraneous signal transduction. The oxidative stress at the site of tumor growth is presumably conveyed by ROS produced by adjacent phagocytic cells (monocyte/macrophages (MO) or neutrophilic granulocytes (GR)). Histamine, an inhibitor of ROS production in phagocytes, is currently used as an adjunct to lymphocyte-activating cytokines (IL-2 and IFN-alpha) with the aim to enhance cytokine efficiency.

Rubin, et al., discusses the use of histamine and other compounds that act as “primers” to treat cancer, including liver cancer. (See U.S. Pat. No. 6,303,660). The primer compound is thought to act by increasing the intracellular levels of cyclic adenosine monophosphate (cAMP) in the normal connective tissue cells within solid tumors. Local administration of the primer to an area containing a tumor is thought to result in a lowering of the interstitial pressure. This reduction in interstitial pressure is also thought to facilitate the uptake of anti-cancer agents.

Given the ravaging effects of liver disease and the only partially successful treatment methods available today, there is a constant demand for improved methods of treating liver disease and reducing hepatic cell injury.

### SUMMARY OF THE INVENTION

The disclosure relates to methods for treating and/or preventing hepatic tissue and cell damage caused by reactive oxygen species in mammals. More specifically, the disclosure relates to the prevention and/or reduction of hepatic tissue and cell damage through the administration of histamine and histamine agonists. In one embodiment, a method for inhibiting and reducing reactive oxygen species (ROS)-mediated oxidative damage to hepatic cells and tissues of a subject is provided. One aspect of the method comprises the step of administering a compound effective to inhibit the production or release of enzymatically produced ROS to a subject suffering from a liver disease caused or exacerbated by ROS-mediated oxidative damage. Although the compositions and methods are applicable to any liver disease, the methods are particularly relevant to the treatment of liver diseases selected from the group consisting of Portal hypertension, Alagille Syndrome, Alpha-1-Antitrypsin Deficiency, Autoimmune Hepatitis, Biliary Atresia, Chronic Hepatitis, Cancer of the Liver, Cancer metastatic to the liver, Cirrhosis, Intrahepatic cholestasis, Hepatic Vein Thrombosis, Hepatic Veno-Occlusive Disease, Hepatolenticular Degeneration, Hepatomegaly, Hepatopulmonary Syndrome, Hepatorenal Syndrome, Liver Cysts, Liver Abscesses, Fatty Liver, Galactosemia, Gilbert's Syndrome, Portal Hypertension, Alcoholic Liver Disease (ALD), Parasitic Liver Diseases, Peliosis Hepatis, Erythrohepatic Porphyria, Hepatic Porphyria, Hepatic Tuberculosis, Primary Biliary Cirrhosis, Primary Sclerosing Cholangitis, Reye's Syndrome, Sarcoidosis, Tyrosinemia, Type I Glycogen Storage Disease, Wilson's Disease, Neonatal Hepatitis, NonAlchoholic SteatoHepatitis, Hemochromatosis, and Zellweger Syndrome.

Another embodiment of the disclosure relates to a method for treating a subject suffering from a disease state wherein phagocyte produced, reactive oxygen species (ROS)-mediated oxidative damage can occur. Aspects of the method comprise identifying a subject with a liver disease in which ROS cause ROS-meditated oxidative damage and administering a compound effective to inhibit the production or release of ROS.

In yet another aspect of the invention, a composition including a compound effective to inhibit the production or release of reactive oxygen species and a hepatotoxic drug is provided. The hepatotoxic drug may be azathioprine, methyldopa, nitrofuratoin, clofibrate, troglizatzone, ibuprofen, allopurinol, indomethacin, leflunomide, acetaminophen, diclofenac, isoniazid, tetracycline, erythromycin, nitrofuratoin, amoxicillin, rifampin, ketoconazole, flucloxacillin, trovafloxacin, sulfonamides, estradiol, iron, glutathione, halothane, isoflurane, captopril, diltiazem, phenytoin, valproic acid, cabamazepine, phenobarbitone, primidone, trazodone, chlorpromazine, quinidine, procainamide, amiodarone, methotrexate, cyclophosphamide, corticosteroid, anabolic steroid, glucocorticoids, pyrazinamide, para-amino salicylic acid, ethionamide, trimethoprim-sulfamethoxazole, pentamidine, zidovudine, dideoxyinosine, penicillins, or cephalosporins.

In another aspect of the invention, herbal preparations are formulated with a compound effective to inhibit the production or release of reactive oxygen species to minimize the hepatotoxic effect of these herbal preparations. Examples of suitable herbs include chapparal (Larrea tridentata), germander (Teucrium chamaedrys), jin bu huan, ma huang, valerian root (Valeriana officinalis), skullcap (Scutellaria galericulata), mistletoe (Viscum species), Jamaican bush tea, senna (Cassia angusfolia), comfrey (Symphytum officinale), and kava root extract.

Advantageously, the compound effective to inhibit the production or release of reactive oxygen species is histamine, histamine receptor agonists, NADPH oxidase inhibitors, serotonin or serotonin agonists. Optionally, the composition further includes an effective amount of a ROS scavenger. The ROS scavenger may be catalase, superoxide dismutase, glutathione peroxidase, or ascorbate peroxidase.

In yet another aspect of the invention, a method of reducing the hepatotoxicity of a drug by administering to an individual taking a hepatotoxic drug an effective dose of a compound effective to inhibit the production or release of ROSs is provided. The compound effective to inhibit the production or release of ROSs may include histamine, histamine receptor agonists, NADPH oxidase inhibitors, serotonin or serotonin agonists.

Optionally, the method further includes the step of administering an effective amount of a ROS scavenger. Advantageously, the step of administering said ROS scavenger results in ROS scavenger catalyzed decomposition of ROS. The scavenger can include catalase, glutathione peroxidase, ascorbate peroxidase, superoxide dismutase, glutathione peroxidase, ascorbate peroxidase, vitamin A, vitamin E, or vitamin C.

The hepatotoxic drug may be azathioprine, methyldopa, nitrofuratoin, clofibrate, or troglizatzone. In one aspect of the invention, the hepatotoxic drug is an anti-arthritic drugs such as ibuprofen, allopurinol, indomethacin, leflunomide, acetaminophen, or diclofenac. In another aspect, the hepatotoxic drug is an antibiotic such as isoniazid, tetracycline, erythromycin, nitrofuratoin, amoxicillin, rifampin, ketoconazole, flucloxacillin, trovafloxacin, or sulfonamides. The hepatotoxic drug may be estradiol, iron, glutathione, halothane, isoflurane, captopril, diltiazem, phenytoin, valproic acid, cabamazepine, phenobarbitone, primidone, trazodone, chlorpromazine; quinidine, procainamide, or amiodarone. The hepatotoxic drug may similarly include a chemotherapeutic agent, corticosteroid, anabolic steroids, or glucocorticoids.

In yet another aspect of the invention, the hepatotoxic drug is a drug used to treat HIV/AIDS patients such as pyrazinamide, para-amino salicylic acid, ethionamide, trimethoprim-sulfamethoxazole, pentamidine, zidovudine, dideoxyinosine, penicillins, and cephalosporins.

A method of reducing the hepatotoxicity of certain herbal preparations is likewise provided. A compound effective to inhibit the production or release of reactive oxygen species are administered to an individual consuming, for example, chapparal (Larrea tridentata), germander (Teucrium chamaedrys), jin bu huan, ma huang, valerian root (Valeriana officinalis), skullcap (Scutellaria galericulata), mistletoe (Viscum species), Jamaican bush tea, senna (Cassia angustfolia), comfrey (Symphytum officinale), and/or kava root extract.

In still another aspect of the present invention, a method of reducing hepatic tissue damage associated with exposure to an environmental or industrial toxin is provided. The method includes administering to a subject in need thereof an effective amount of a compound effective to inhibit the production or release of ROS. Advantageously, the compound effective to inhibit the production or release of ROS includes histamine, histamine receptor agonists, NADPH oxidase inhibitors, serotonin and serotonin agonists. Optionally, the method may include a further step of administering an effective amount of a ROS scavenger. Preferably, the step of administering the ROS scavenger results in ROS scavenger catalyzed decomposition of ROS. The scavenger may be catalase, glutathione peroxidase, ascorbate peroxidase, superoxide dismutase, glutathione peroxidase, ascorbate peroxidase, vitamin A, vitamin E, or vitamin C. The environmental toxin may include a poisonous mushroom from the Amanita, Galerina, or Gyromitra genus. Additionally, the environmental or industrial toxin may include cigarette smoke, pesticides, food additives and/or preservatives, heavy metal, organic solvents, or industrial cleaners, particularly those containing chlorinated solvents.

### DETAILED DESCRIPTION OF THE PREFERRED EMBODIMENT

The disclosure below relates to compositions and methods for preventing and reducing hepatic cellular and tissue damage caused by reactive oxygen species (ROS).

The liver plays an essential role in a variety of metabolic functions. Diseases of the liver typically have serious consequences for the person afflicted, ranging from a morbidity to mortality. Examples of liver diseases include: Portal hypertension, Alagille Syndrome, Alpha-1-Antitrypsin Deficiency, Autoimmune Hepatitis, Biliary Atresia, Chronic Hepatitis, Primary Cancer of the Liver, Cancer metastatic to the liver, Cirrhosis, Intrahepatic cholestasis, Hepatic Vein Thrombosis, Hepatic Veno-Occlusive Disease, Hepatolenticular Degeneration, Hepatomegaly, Hepatopulmonary Syndrome, Hepatorenal Syndrome, Liver Cysts, Liver Abscesses, Fatty Liver, Galactosemia, Gilbert's Syndrome, Portal Hypertension, Alcoholic Liver Disease (ALD), Parasitic Liver Diseases, Peliosis Hepatis, Erythrohepatic Porphyria, Hepatic Porphyria, Hepatic Tuberculosis, Primary Biliary Cirrhosis, Primary Sclerosing Cholangitis, Reye's Syndrome, Sarcoidosis, Tyrosinemia, Type I Glycogen Storage Disease, Wilson's Disease, Neonatal Hepatitis, NonAlchoholic SteatoHepatitis, Hemochromatosis, and Zellweger Syndrome. “Alcoholic Liver Disease” (ALD), as used herein, includes without limitation, alcoholic fatty liver, alcoholic hepatitis, alcoholic liver cirrhosis, and fibrosis.

Recent work has indicated that these and other liver diseases may be caused or exacerbated by ROS. ROS can have direct effects on various cells within the liver parenchema leading to apoptosis. Another possible mechanism by which these molecules can damage hepatic cells and tissue may be related to the effect ROS have on actuator cells of the immune system. For example, ROS evolved from monocytes and other sources have been shown to effectively suppress the activation and activity of NK cells and T-cells.

The effects of ROS production are many faceted. ROS are known to cause apoptosis in NK cells. ROS are also known to cause anergy and/or apoptosis in T-cells. The mechanisms by which ROS cause these effects are not yet fully understood. Nevertheless, some commentators believe that ROS cause cell death by disrupting cellular membranes and by changing the pH of cellular pathways critical for cell survival and also by direct damaging effects on DNA.

The compounds which reduce the amount of ROS produced and released in an individual and the methods disclosed below are directed to the reduction and prevention of ROS mediated damage of hepatic cells and tissue. In preferred embodiments, various histamine and histamine-related compounds are used to achieve a beneficial reduction or inhibition of enzymatic ROS production and release or the net concentration thereof. The term “histamine” as used herein incorporates a variety of histamine and histamine related compounds. For example, histamine, the dihydrochloride salt form of histamine (histamine dihydrochloride), histamine diphosphate, other histamine salts, esters, or prodrugs, and histamine receptor agonists are to be included. Also included within the meaning of the term “histamine” are histamine binding mimics and NADPH oxidase inhibitors.

The administration of compounds that induce the release of endogenous histamine from a patient's own tissue stores is also included within the scope of the present disclosure. Such compounds include IL-3, retinoids, and allergens. Other ROS production and release inhibitory compounds such as NADPH oxidase inhibitors like diphenyleneiodonium can also be used with the disclosed methods, as can serotonin and 5HT-receptor agonists.

The compositions and methods disclosed herein also encompass the administration of a variety of ROS scavengers. Known scavengers of ROS include the enzymes catalase, superoxide dismutase (SOD), glutathione peroxidase and ascorbate peroxidase. Additionally, vitamins A, E, and C are known to have scavenger activity. Minerals such as selenium and manganese can also be efficacious in combating ROS-mediated damage. The scope of the methods disclosed herein includes the administration of the compounds listed and those compounds with similar ROS inhibitor activity. The compositions and methods disclosed herein also provide an effective means for preventing and/or inhibiting the release of enzymatically generated ROS in excessive amounts or at inappropriate times or locations.

Compounds and methods for treating hepatic disease states that are complicated by the detrimental release of ROS within a host or subject are provided. The liver is responsible for many essential functions in the body. Because of these activities, the liver is exposed to a wide variety of insults and is therefore one of the most frequently injured organs in the body. The impairment of these vital functions by hepatic disease can lead to very serious consequences. Liver damage has been linked to a number of sources. Hepatitis, or inflammation of the liver, may be caused by infections with viruses, bacteria, fungi, or protozoa or may result from exposure to toxins such as alcohol, drugs, chemical poisons, or other environmental toxins. Vascular and metabolic disorders, neoplastic disease, and/or the liver's involvement in extrahepatic disorders such as autoimmune disease have similarly been linked to damage of hepatic tissue. Examples of autoimmune diseases wherein liver tissue becomes comprised include lupus erythematosus and rheumatoid arthritis. Additionally, in patients suffering from heart failure, the liver is often damaged from congestion, scarring, and ascites formation.

In the case of industrial and environmental toxins, primary disease is caused by the ingestion, injection, or inhalation of a toxic substance which adversely affects the liver. For example, herbicides, such as paraquat, are associated with an increased incidence of liver damage. Similarly, the ingestion of certain genuses of mushrooms can result in grave damage to hepatic cells and even death. Such mushrooms include the Amanita and the Galerina genuses which contain amatoxin, the substance responsible for hepatic and renal destruction. Hepatic necrosis, similar to that produced by acetaminophen over dosage, is the primary toxic manifestation. Mushrooms from the genus Gyromitra have likewise been associated with hepatotoxicity and hepatorenal syndrome in severe cases. Examples of environmental and industrial toxins which cause damage to hepatic tissue include, without limitation, cigarette smoke, industrial cleaners, diethanoloamine, sodium laurel sulfate, propylene glycol, pesticides such as DDT and mirex, food additives and preservatives, heavy metals, organic solvents such as formaldehyde and bromobenzene, and chlorinated solvents such as dioxins, flurans, TCE, PCE, DCE, tetrachloroethylene, carbon tetrachloride, and vinyl chloride.

As will be described in greater detail below, liver toxins also include many common drugs, such as acetaminophen, anabolic steroids, chemotherapy drugs, some antibiotics, glucocorticoids, anaesthetics, parasite control drugs, and phenylbutazone. Certain anticonvulsants are associated with hepatopathy such as phenobarbital, primidone, and phentoin. Damage to liver tissue results, at least in part, by the detrimental release of ROS within a host or subject in response to such insults. Accordingly, compositions and methods for treating damage to liver tissue caused by exposure to toxic substances are provided. Specifically, the administration of a ROS production and release inhibiting compound is useful for the reduction in trauma to hepatic cells and tissues following exposure to industrial and/or environmental toxins.

Numerous medications have been associated with damage to the liver. Approximately 10% of all cases of hepatitis in young adults and 40% of cases in patients older than 50 are caused by medications. As used herein, “hepatotoxic drugs” include any substance or substances which act upon the liver to cause tissue damage. Examples of hepatotoxic drugs include, without limitation, anti-diabetic and immunosuppressive drugs such as azathioprine, methyldopa, nitrofuratoin, clofibrate, and troglizatzone; anti-arthritic drugs such as ibuprofen, allopurinol, indomethacin, leflunomide, acetaminophen, diclofenac; antibiotics such as isoniazid, tetracycline, erythromycin, nitrofuratoin, amoxicillin, rifampin, ketoconazole, flucloxacillin, trovafloxacin, and sulfonamides; estradiol; iron; vitamin A; glutathione; certain anesthetics such as halothane and isoflurane; anti-hypertensive drugs including captopril and diltiazem; anti-convulsants and anti-depressants such as phenytoin, valproic acid, cabamazepine, phenobarbitone, primidone, and trazodone; anti-psychotics such as chlorpromazine; anti-arrhythmics like quinidine, procainamide, and amiodarone; chemotherapeutics such as methotrexate and cyclophosphamide; steroids including corticosteroid, anabolic steroids, and glucocorticoids; and drugs commonly used in HIV/AIDS patients such as pyrazinamide, para-amino salicylic acid, ethionamide, trimethoprim-sulfamethoxazole, pentamidine, zidovudine, dideoxyinosine, penicillins, and cephalosporins. Additionally, herbal preparations such as chapparal (Larrea tridentata); germander (Teucrium chamaedrys); certain Chinese herbs including jin bu huan and ma huang; valerian root (Valeriana officinalis); skullcap (Scutellaria galericulata); mistletoe (Viscum species); herbal teas such as Jamaican bush tea (pyrrolizidine alkaloids); senna (Cassia angustfolia); comfrey (Symphytum officinale); and kava root extract are known hepatotoxins.

Compositions and methods for minimizing the hepatotoxicity of certain drugs are provided. With many diseases such as HIV/AIDS, the treatment can seem as onerous as the disease. In the case of HIV infection, the drugs designed to reduce viral loads often compromise liver cells and can result in grave consequences. Accordingly, ROS inhibiting or scavenging compounds can be administered to an individual who is concurrently taking a drug or drugs which cause hepatotoxic side effects to mitigate damage to liver cells caused by the hepatotoxic drug. In one embodiment, an individual taking a hepatotoxic drug is administered an effective amount of a ROS inhibiting compound or scavenger separately or as a single formulation with the hepatotoxic drug. The ROS inhibiting compound or scavenger and hepatotoxic drug may be given substantially simultaneously or within various time durations of each other. The administration can be by either local or by systemic injection or infusion. Other methods of administration may also be suitable, such as by oral route.

The administration of an ROS inhibitor or scavenger is likewise useful for ameliorating damage to liver tissue caused or exacerbated by bacterial, fungal, viral, or protozoan infections. Helicobacter and Leptospirosis are just two examples of a species of pathogenic bacteria which invades the liver and causes tissue damage. Blasomycosis, histoplasmosis, and coccidiomycosis are examples of fungal infections which attack liver tissue. Systemic infections, such as tuberculosis, candidiasis, and toxoplasmosis, often spread to the liver and can cause damage to the liver tissue. Moreover, nearly all blood-born infections will inevitably involve the liver.

Accordingly, in one aspect of the present invention, compounds and methods for minimizing damage to liver tissue associated with bacterial, fungal, viral, or protozoal infections are provided. ROS production and release inhibiting compounds are administered alone or in combination with an antibiotic. As used herein, the term “antibiotic” includes any antibacterial, antifungal, or anti-protozoal compound. When administered in combination with antibiotics, the ROS production and release inhibiting compound of the present invention can be administered separately or as a single formulation with the antibiotic. If administered separately, the ROS production and release inhibiting compound should be given in a temporally proximate manner such that the amelioration of damage to liver tissue is enhanced. In one embodiment, the ROS production and release inhibiting compound and antibiotic are given within one week of each other. In another embodiment, the ROS production and release inhibiting compound and antibiotic are given within twenty-four hours of each other. In yet another embodiment, the ROS production and release inhibiting compound and antibiotic are given within one hour of each other. The administration can be by either local or by systemic injection or infusion. Other methods of administration may also be suitable such as oral administration.

In yet another embodiment, compositions and methods for treating liver diseases secondary to other disease etiologies are provided. Because of the close proximity of the pancreas to the liver and the bile ducts, acute pancreatitis often leads to hepatitis. Similarly, chronic inflammation of the bowel allows portal absorption of toxic intestinal products and bacteria which can compromise the tissue of the liver. Shock, anemia, and congestive heart failure result in severe loss of blood circulation to the liver and lack of oxygen, which can likewise lead to damage to liver tissue. Similarly, while primary cancer of the liver is rare, it is common for cancer to spread to the liver as a secondary metastatic cancer from the colon, lungs, breasts, or other parts of the body. Therefore, compositions comprising an ROS inhibiting compound or scavenger are useful for treating liver diseases which are secondary to other diseases. In one embodiment, a patient suffering from acute pancreatitis is administered an effective dose of an ROS inhibiting compound or scavenger to prevent damage to hepatic cells. In another embodiment, an individual with metastatic cancer of the liver is administered an effective dose of an ROS inhibiting compound or scavenger with or without chemotherapeutic agents to minimize damage to the liver.

The administration of the disclosed compounds can be alone or in combination with other compounds effective at treating various hepatic disease states. For example, histamine can be used to treat a patient suffering from nonalchoholic steatohepatitis (NASH). Further, the disclosed methods and compounds can be used with standard NASH treatment regimes, which usually comprise a low fat, low calorie diet along with insulin or medications to lower blood sugar for obese patients. For patients with NASH who are not overweight and not diabetic, a low fat diet is often recommended. Also, as discussed above, individuals presenting with metastatic cancer of the liver are administered an effective dose of an ROS inhibiting compound or scavenger along with standard chemotherapy and/or radiation protocols. In the case of viral infections of the liver, a subject can be administered an anti-viral therapy concurrently with the administration of an ROS inhibiting compound or scavenger to minimize hepatic cell injury. For example, an ROS inhibiting compound or scavenger can be used to treat an individual presenting with Hepatitis C along with standard hepatitis treatment regimes including the administration of ribavirin or pegylated INF-α.

Similarly, the disclosed methods and compounds are useful for the treatment of alcoholic liver diseases. Alcohol abuse is a leading cause of morbidity and mortality throughout the world. It is estimated that in the United States as many as 10% of men and 3% of women may suffer from persistent problems related to the use of alcohol. Alcohol affects many organ systems of the body, but perhaps most notably affected is the liver because almost all ingested alcohol must be metabolized in the liver. Alcohol abuse generally leads to three pathologically distinct liver diseases: fatty liver (steatosis), alcoholic hepatitis, and cirrhosis. Fatty liver is characterized by the accumulation of fat within hepatocytes, the predominant cell type in the liver. Alcohol can also cause acute and chronic hepatitis. Alcoholic hepatitis can lead to liver scarring and cirrhosis, and very frequently occurs in alcoholics who already have cirrhosis of the liver. Finally, liver cirrhosis resulting from alcohol abuse is characterized by the development of widespread nodules in the liver combined with fibrosis and can lead to end-stage liver disease. Alcohol-related cirrhosis is one of the ten leading causes of death in the United States. Some of the complications of cirrhosis are jaundice, ascites, edema, bleeding esophageal varices, blood coagulation abnormalities, coma and death. Thus, in one embodiment, the disclosed methods and compositions are administered to a subject suffering from alcoholic liver disease (ALD). A subject suffering from ALD is identified and administered an effective dose of an ROS inhibiting or scavenging compound.

The use of the ROS inhibiting or scavenging compounds can be by any of a number of methods well known to those of skill in the art. For oral administration, the ROS inhibiting or scavenging compounds may be incorporated into a tablet, aqueous or oil suspension, dispersible powder or granule, microbead, emulsion, hard or soft capsule, syrup or elixir. The compositions may be prepared according to any method known in the art for the manufacture of pharmaceutically acceptable compositions and such compositions may contain one or more of the following agents: sweeteners, flavoring agents, coloring agents and preservatives. Tablets containing the active ingredients in admixture with non-toxic pharmaceutically acceptable excipients suitable for tablet manufacture are acceptable. “Pharmaceutically acceptable” means that the agent should be acceptable in the sense of being compatible with the other ingredients of the formulation (as well as non-injurious to the individual). Such excipients include inert diluents such as calcium carbonate, sodium carbonate, lactose, calcium phosphate or sodium phosphate; granulating and disintegrating agents, such as corn starch and alginic acid; binding agents such as starch, gelatin or acacia; and lubricating agents such as magnesium stearate, stearic acid or talc. Tablets may be uncoated or may be coated with known techniques to delay disintegration and absorption in the gastrointestinal tract and thereby provide a sustained action over a longer period of time. For example, a time delay material such as glyceryl monostearate or glyceryl stearate alone or with a wax may be employed.

In another preferred embodiment, tablets, capsules or microbeads are coated with an enteric coating which prevents dissolution in the acidic environment of the stomach. Instead, this coating dissolves in the small intestine at a more neutral pH. Such enteric coated compositions are described by Bauer et al., Coated Pharmaceutical Dosage Forms: Fundamentals, Manufacturing Techniques, Biopharmaceutical Aspects, Test Methods and Raw Materials, CRC Press, Washington, DC, 1998, the entire contents of which are hereby incorporated by reference.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

Aqueous suspensions may contain the ROS inhibiting or scavenging compounds of the invention in admixture with excipients for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions may be formulated by suspending the active ingredient in a vegetable oil, such as arachis oil, olive oil, sesame oil or coconut oil, or in a mineral oil such as liquid paraffin. The oil suspension may contain a thickening agent, such as beeswax, hard paraffin or cetyl alcohol. Sweetening agents, such as those set forth above, and flavoring agents may be added to provide a palatable oral preparation. These compositions may be preserved by an added antioxidant such as ascorbic acid. Dispersible powders and granules of the compounds of the invention, suitable for preparation of an aqueous suspension by the addition of water, provide the active ingredient in admixture with a dispersing or wetting agent, a suspending agent, and one or more preservatives. Additional excipients, for example sweetening, flavoring and coloring agents, may also be present.

Syrups and elixirs may be formulated with sweetening agents, such as glycerol, sorbitol or sucrose. Such formulations may also contain a demulcent, a preservative, a flavoring or a coloring agent.

The use of the ROS inhibiting or scavenging compounds can also be accomplished via parenteral delivery through subcutaneous, intravenous, intraperitoneal, or intramuscular injection. The compounds can be administered in an aqueous solution with or without a surfactant such as hydroxypropyl cellulose. Dispersions are also contemplated such as those utilizing glycerol, liquid polyethylene glycols, and oils. Injectable preparations can include sterile aqueous solutions or dispersions and powders that can be diluted or suspended in a sterile environment prior to use. Carriers such as solvents or dispersion media contain water, ethanol polyols, vegetable oils and the like can also be added to the disclosed compounds. Coatings such as lecithins and surfactants can be used to maintain the proper fluidity of the composition. Isotonic agents such as sugars or sodium chloride can be added, as well as products intended to delay absorption of the active compounds such as aluminum monostearate and gelatin. Sterile injectable solutions are prepared according to methods well known to those of skill in the art and can be filtered prior to storage and/or use. Sterile powders can be vacuum or freeze dried from a solution or suspension. Sustained or controlled release preparations and formulations can also be used with the disclosed methods. Typically the materials used with the disclosed methods and compositions are pharmaceutically acceptable and substantially non-toxic in the amounts employed.

The disclosed compounds can also be administered by inhalation. In this administration route, histamine can be dissolved in water or some other pharmaceutically acceptable carrier liquid for inhalation, or provided as a dry powder, and then introduced into a gas or powder that is then inhaled by the patient in an appropriate volume so as to provide that patient with a measured amount of histamine. Examples of the administration of a therapeutic composition via inhalation are described in U.S. Pat. Nos. 6,418,926; 6,387,394; 6,298,847; 6,182,655; 6,132,394; and 6,123,936, which are hereby incorporated by reference.

Infusion devices can be used to deliver the disclosed compounds. Suitable devices include syringe pumps, auto injector systems, implantable pumps, implantable devices, and minipumps. Exemplary devices include the Ambulatory Infusion Pump Drive, Model 30, available from Microject Corp., Salt Lake City, Utah, and the Baxa Syringe Infuser, available from Baxa Corporation, Englewood, Colo. Any device capable of delivering the disclosed compounds in accordance with the methods disclosed herein can be used.

Suitable infusion devices preferably have an effective amount of histamine, histamine agonist, histamine salt, histamine prodrug, NADPH-oxidase inhibitor, histamine dihydrochloride, histamine phosphate, serotonin, a 5HT agonist, a histamine receptor agonist, histamine receptor binding mimic, or a substance which induces the release of an effective therapeutic amount of endogenous histamine contained therein. The device can be pre-loaded with the desired substance during manufacture, or the device can be filled with the substance just prior to use. Pre-filled infusion pumps and syringe pumps are well known to those of skill in the art. The active substance can be part of a formulation which includes a controlled release carrier, if desired. A controller is used with the device to control the rate of administration and the amount of substance to be administered. The controller can be integral with the device or it can be a separate entity. It can be pre-set during manufacture, or set by the user just prior to use. Such controllers and their use with infusion devices are well known to those of skill in the art.

Controlled release vehicles are well known to those of skill in the pharmaceutical sciences. The technology and products in this art are variably referred to as controlled release, sustained release, prolonged action, depot, repository, delayed action, retarded release and timed release; the words “controlled release” as used herein is intended to incorporate each of the foregoing technologies.

Numerous controlled release vehicles are known, including biodegradable or bioerodable polymers such as polylactic acid, polyglycolic acid, and regenerated collagen. Known controlled release drug delivery devices include creams, lotions, tablets, capsules, gels, microspheres, liposomes, ocular inserts, minipumps, and other infusion devices such as pumps and syringes. Implantable or injectable polymer matrices, and transdermal formulations, from which active ingredients are slowly released are also well known and can be used in the disclosed methods.

In one embodiment, the disclosed compounds are administered through a topical delivery system. The controlled release components described above can be used as the means to delivery the disclosed compounds. A suitable topical delivery system comprises the disclosed compounds in concentrations taught herein, a solvent, an emulsifier, a pharmaceutically acceptable carrier material, penetration enhancing compounds, and preservatives. Examples of topically applied compositions include U.S. Pat. Nos. 5,716,610 and 5,804,203, which are hereby incorporated by reference. The compositions can further include components adapted to improve the stability or effectiveness of the applied formulation, such as preservatives, antioxidants, skin penetration enhancers and sustained release materials. Examples of such components are described in the following reference works hereby incorporated by reference: Martindale—The Extra Pharmacopoeia (Pharmaceutical Press, London 1993) and Martin (ed.), Remington's Pharmaceutical Sciences.

Controlled release preparations can be achieved by the use of polymers to complex or absorb the ROS inhibiting or scavenging compound. The controlled delivery can be exercised by selecting appropriate macromolecule such as polyesters, polyamino acids, polyvinylpyrrolidone, ethylenevinyl acetate, methylcellulose, carboxymethylcellulose, and protamine sulfate, and the concentration of these macromolecule as well as the methods of incorporation are selected in order to control release of active compound.

Hydrogels, wherein the ROS inhibiting or scavenging compound is dissolved in an aqueous constituent to gradually release over time, can be prepared by copolymerization of hydrophilic mono-olefinic monomers such as ethylene glycol methacrylate. Matrix devices, wherein the ROS inhibiting or scavenging compound is dispersed in a matrix of carrier material, can be used. The carrier can be porous, non-porous, solid, semi-solid, permeable or impermeable. Alternatively, a device comprising a central reservoir of the ROS inhibiting or scavenging compound surrounded by a rate controlling membrane can be used to control the release of the ROS inhibiting or scavenging compound. Rate controlling membranes include ethylene-vinyl acetate copolymer or butylene terephthalate/polytetramethylene ether terephthalate. Use of silicon rubber depots are also contemplated.

Controlled release oral formulations are also well known. In one embodiment, the active compound is incorporated into a soluble or erodible matrix, such as a pill or a lozenge. Such formulations are well known in the art. An example of a lozenge used to administer pharmaceutically active compounds is U.S. Pat. No. 5,662,920, which is hereby incorporated by reference. In another example, the oral formulations can be a liquid used for sublingual administration. An example of pharmaceutical compositions for liquid sublingual administration of the disclosed compounds are taught in U.S. Pat. No. 5,284,657, which is hereby incorporated by reference. These liquid compositions can also be in the form a gel or a paste. Hydrophilic gums, such as hydroxymethylcellulose, are commonly used. A lubricating agent such as magnesium stearate, stearic acid, or calcium stearate can be used to aid in the tableting process.

For the purpose of parenteral administration, ROS inhibiting or scavenging compounds can be combined with distilled water, preferably buffered to an appropriate pH and having appropriate (e.g., isotonic) salt concentrations. The compounds of the present invention can also be provided as a liquid or as a powder that is reconstituted before use. They can be provided as prepackaged vials, syringes, or injector systems.

The disclosed compounds, such as histamine, can also be provided in septum-sealed vials in volumes ranging from about 0.5 to 100 ml for administration to an individual. In a preferred embodiment, the vials contain volumes of 0.5, 1, 3, 5, 6, 8, 10, 20, 50 and 100 ml. The vials are preferably sterile. The vials can optionally contain an isotonic carrier medium and/or a preservative. Any desired amount of histamine can be used to give a desired final histamine concentration. In a preferred embodiment, the ROS inhibiting or scavenging compound concentration is between about 0.01 mg/ml and 100 mg/ml. More preferably, the ROS inhibiting or scavenging compound concentration is between about 0.1 and 50 mg/ml. Most preferably, the ROS inhibiting or scavenging compound concentration is between about 1 mg/ml and 10 mg/ml. At the lower end of the volume range, it is preferred that individual doses are administered, while at the higher end it is preferred that multiple doses are administered.

In a preferred embodiment, transdermal patches, steady state reservoirs sandwiched between an impervious backing and a membrane face, and transdermal formulations, can also be used to deliver ROS inhibiting or scavenging compounds. Transdermal administration systems are well known in the art. Occlusive transdermal patches for the administration of an active agent to the skin or mucosa are described in U.S. Pat. Nos. 4,573,996, 4,597,961 and 4,839,174, which are hereby incorporated by reference. One type of transdermal patch is a polymer matrix in which the active agent is dissolved in a polymer matrix through which the active ingredient diffuses to the skin. Such transdermal patches are disclosed in U.S. Pat. Nos. 4,839,174, 4,908,213 and 4,943,435, which are hereby incorporated by reference. In one embodiment, the steady state reservoir carries doses of histamine and other ROM production and release inhibitory compounds in doses from about 0.2 to 5 mg per day.

Present transdermal patch systems are designed to deliver smaller doses over longer periods of time, up to days and weeks. A preferred delivery system for the disclosed compounds would specifically deliver an effective dose of histamine in a range of between about 2 and 60 minutes, depending upon the dose, with a preferred dose being delivered within about 20-30 minutes. These patches allow rapid and controlled delivery of a compound which inhibits or scavenges ROS. A rate-controlling outer microporous membrane, or micropockets of the disclosed compounds dispersed throughout a silicone polymer matrix, can be used to control the release rate. Such rate-controlling means are described in U.S. Pat. No. 5,676,969, which is hereby incorporated by reference. In another preferred embodiment, the histamine or other ROM inhibiting or scavenging compound is released from the patch into the skin of the patient in about 20-30 minutes or less. In a preferred embodiment, the compound is released from the patch at a rate of between about 0.025 mg to 0.5 mg per minute for a dose of between about 0.2 mg and 5 mg per patch.

These transdermal patches and formulations can be used with or without use of a penetration enhancer such as dimethylsulfoxide (DMSO), combinations of sucrose fatty acid esters with a sulfoxide or phosphoric oxide, or eugenol. The use of electrolytic transdermal patches is also within the scope of the methods disclosed herein. Electrolytic transdermal patches are described in U.S. Pat. Nos. 5,474,527, 5,336,168, and 5,328,454, the entire contents of which are hereby incorporated by reference.

In another embodiment transmucosal patches can be used to administer the disclosed compounds. An example of such a patch is found in U.S. Pat. No. 5,122,127, which is hereby incorporated by reference. The described patch comprises a housing capable of enclosing a quantity of therapeutic agent where the housing is capable of adhering to mucosal tissues, for example, in the mouth. A drug surface area of the device is present for contacting the mucosal tissues of the host. The device is designed to deliver the drug in proportion to the size of the drug/mucosa interface. Accordingly, drug delivery rates can be adjusted by altering the size of the contact area.

The housing is preferably constructed of a material which is nontoxic, chemically stable, and non-reactive with the disclosed compounds. Possible construction materials include: polyethylene, polyolefins, polyamides, polycarbonates, vinyl polymers, and other similar materials known in the art. The housing can contain means for maintaining the housing positioned against the mucosal membrane. The housing can contain a steady state reservoir positioned to be in fluid contact with mucosal tissue.

Steady state reservoirs for use with the disclosed compounds delivery a suitable dose of those compounds over a predetermined period of time. Compositions and methods of manufacturing compositions capable of absorption through the mucosal tissues are taught in U.S. Pat. No. 5,288,497, which is hereby incorporated by reference. One of skill in the art could readily include the disclosed compounds and related compositions.

The steady state reservoirs for use with the disclosed compounds are composed of compounds known in the art to control the rate of drug release. In one embodiment, the transmucosal patch delivers a dose of a ROS inhibiting or scavenging compound over a period of time from about 2 to 60 minutes. The steady state reservoir contained within the housing carries doses of histamine and other ROS production and release inhibitory or scavenging compounds in doses from about 0.2 to 100 mg per patch. Transdermal patches that can be worn for several days and that release the disclosed compounds over that period of time are also contemplated. The reservoirs can also contain permeation or penetration enhancers, as discussed above, to improve the permeability of the disclosed compounds across the mucosal tissue.

Another method to control the release of the disclosed compounds is to incorporate the ROS inhibiting or scavenging compound into particles of a polymeric material such as polyesters, polyamino acids, hydrogels, poly lactic acid, or ethylene vinylacetate copolymers.

Alternatively, instead of incorporating the ROS inhibiting or scavenging compounds into these polymeric particles, the disclosed compounds are entrapped in microcapsules prepared, for example, by coacervation techniques, or by interfacial polymerization, for example hydroxymethylcellulose or gelatin-microcapsules, respectively, or in colloidal drug delivery systems, for example, liposomes, albumin microspheres, microemulsions, nanoparticles, and nanocapsules, or in macroemulsions. Such technology is well known to those of ordinary skill in pharmaceutical sciences.

Preferably, the compounds that inhibit or scavenging ROS are injected, infused, or released into the patient at a rate of from about 0.025 to 1.0 mg/min. A rate of about 0.1 mg/min is preferred. The disclosed compounds are preferably administered over a period of time ranging from about 1, 3 or 5 minutes to about 30 minutes, with an upper limit of about 20 minutes being preferred, such that the total daily adult dose of ROS inhibiting or scavenging compound ranges from between about 0.4 to about 100.0 mg, with about 0.5 to about 20.0 mg being preferred. Compounds of the present invention, such as histamine, administered over longer periods of time, i.e., longer than about 30 minutes, have been found to result in decreased or lack of efficacy, while rapid administration over less than 1-3 minutes can cause more pronounced and serious side effects, which include anaphylaxis, heart failure, bronchospasm, pronounced flushing, discomfort, increased heart rate and respiratory rate, hypotension, and severe headache.

In another embodiment, an ROS inhibiting compound at approximately 0.2 to 2.0 mg or 3-200 μg/kg, in a pharmaceutically acceptable form can be administered. ROS scavenging compounds can also be administered in combination with the ROS production and release inhibitory compounds described above. When the ROS inhibiting or scavenging compound is administered orally, the composition can be formulated as a tablet comprising between 10 mg to 2 grams of active ingredient. A tablet can include 10, 20, 50, 100, 200, 500, 1,000, or 2,000 milligrams of ROS inhibiting or scavenging compound. Preferably, the amount of ROS inhibiting or scavenging compound in a tablet is 100 mg. In some embodiments, the composition includes histamine protectors such as diamine oxidase inhibitors, monoamine oxidase inhibitors and n-methyl transferases.

The treatment can also include periodically boosting patient blood ROS inhibiting or scavenging compound levels by administering 0.2 to 2.0 mg or 3-200 μg/kg of the disclosed compounds injected or ingested 1, 2, or more times per day over a period of one to two weeks at regular intervals, such as daily, bi-weekly, or weekly in order to establish blood levels of ROS inhibiting or scavenging compound at a beneficial concentration such that ROS production and release is inhibited. The treatment is continued until the causes of the patient's underlying disease state is controlled or eliminated.

Administration of each dose of ROS inhibiting or scavenging compound can occur from once a day to up to about four times a day, with twice a day being preferred. Administration can be subcutaneous, intravenous, intramuscular, intraocular, oral, transdermal, intranasal, or rectal and can utilize direct hypodermic or other injection or infusion means, or can be mediated by a controlled release mechanism of the type disclosed above. Any controlled release vehicle or infusion device capable of administering a therapeutically effective amount of the disclosed compounds over a period of time ranging from about 1 to about 90 minutes can be used. In a preferred embodiment, intranasal delivery is accomplished by using a solution of ROS inhibiting or scavenging compound in an atomizer or nebulizer to produce a fine mist which is introduced into the nostrils. For rectal delivery, ROS inhibiting or scavenging compound is formulated into a suppository using methods well known in the art.

Compounds that scavenge ROS can be administered in an amount of from about 0.1 to about 20 mg/day; more preferably, the amount is from about 0.5 to about 8 mg/day; more preferably, the amount is from about 0.5 to about 8 mg/day; and even more preferably, the amount is from about 1 to about 5 mg/day. Nevertheless, in each case, the dose depends on the activity of the administered compound. The foregoing doses are appropriate for the enzymes listed above that include catalase, superoxide dismutase (SOD), glutathione peroxidase and ascorbate peroxidase. Appropriate doses for any particular host can be readily determined by empirical techniques well known to those of ordinary skill in the art.

Non-enzymatic ROS scavengers can be administered in amounts empirically determined by one of ordinary skill in the art. For example, vitamins A and E can be administered in doses from about 1 to 5000 IU per day. Vitamin C can be administered in doses from about 1 μg to 10 gm per day. Minerals such as selenium and manganese can be administered in amounts from about 1 picogram to 1 milligram per day. These compounds can also be administered as a protective or preventive treatment for ROS mediated disease states.

In addition to histamine, histamine dihydrochloride, histamine phosphate, other histamine salts, esters, congeners, prodrugs, and H₂ receptor agonists, the use of serotonin, 5HT agonists, and compounds which induce release of histamine from the patient's own tissues is also included within the disclosed methods. Retinoic acid, other retinoids such as 9-cis-retinoic acid and all-trans-retinoic acid, IL-3 and ingestible allergens are compounds that are known to induce the release of endogenous histamine. These compounds can be administered to the patient by oral, intravenous, intramuscular, subcutaneous, and other approved routes. The rate of administration should result in a release of endogenous histamine resulting in a blood plasma level of histamine of about 20 nmol/dl.

Administration of each dose of a compound which induces histamine release can occur from once per day to up to about four times a day, with twice per day being preferred. Administration can be subcutaneous, intravenous, intramuscular, intraocular, oral, or transdermal, and can incorporate a controlled release mechanism of the type disclosed above. Any controlled release vehicle capable of administering a therapeutically effective amount of a compound which induces histamine release over a period of time ranging from about one to about thirty minutes can be used. Additionally, the compounds, compositions, and formulations of the present invention can be administered quantum sufficiat.

The following examples teach various methods for treating hepatic disease with the disclosed ROS production and release inhibiting compounds. These examples are illustrative only and are not intended to limit the scope of the claims. The treatment methods described below can be optimized using empirical techniques well known to those of ordinary skill in the art. Moreover, artisans of ordinary skill would be able to use the teachings described in the following examples to practice the full scope of the claims. Although it is stated in the examples that the administration of an ROS inhibiting compound or scavenger may be given in a single dose, it is obvious that the compounds can be distributed over longer periods of time. Moreover, the daily dose can be administered as a single dose or it can be divided into several doses.

### EXAMPLES

### Example 1

### Analysis of the Effect of Histamine Dihydrochloride on the Protection Against Early Alcohol-induced Liver Injury in a Rat Model

Liver disease leading to hepatitis or inflammation of the liver may be caused by a variety of factors that include infectious agents and toxins. The two most common causes are viral infection and chronic alcohol abuse and in both cases the disease manifests in a similar fashion. The following data strongly supports the hypothesis that free radicals generated from NADPH oxidase in hepatic Kupffer cells and infiltrating leukocytes play a predominant role in the pathogenesis of early alcohol-induced hepatitis. In this study, we investigated the effect of histamine treatment of early alcohol induced liver injury in a rat model.

Materials and Methods

Animals and Treatments. Female Wistar rats weighing between 200 and 275 g were fed ad libitum continuously for up to 4 weeks a liquid diet (Dyets, Bethlehem, Pa. #710034) in which 35% of the calories were from corn oil, 23% were from protein, 5% were from vitamins and minerals, 11% were from maltose-dextrin, and 36% were from ethanol. An isocaloric maltose-dextrin diet (Dyets, #710270) was used for control. Rats were given a single dose of ethanol (5 g/kg body weight intragastrically) using an 18-gauge oral biomedical device (Popper & Sons, Inc., New Hyde Park, N.Y.) diluted in PBS (pH 7.2; GIBCO Laboratories Life Technologies Inc., Grand Island, N.Y.) every 24 hours. Control animals were gavaged with PBS alone. Either histamine dihydrochloride (0.5 mg/kg or 5.0 mg/kg; Sigma Chemical Co., St. Louis, Mo.) or vehicle (PBS, pH 7.4; GIBCO Laboratories Life Technologies Inc., Grand Island, N.Y.) was administered by subcutaneous injection twice daily for 4 weeks. Body weights of each rat were measured daily. Rats were housed in a pathogen-free facility and the institutional animal care and use committee approved the surgical procedures used in this study.

Blood Collection and Enzymatic assays. Blood was collected by cardiac puncture at necropsy after 2 and 4 wk of ethanol treatment and centrifuged. Serum was stored at −20° C. until it was assayed for alanine aminotransferase (ALT) and aspartate aminotransferase (AST) by Sigma diagnostic kits (Sigma Chemical Co., St. Louis, Mo.).

Pathological Evaluation. After 2 and 4 weeks of ethanol treatment, livers were formalin fixed, embedded in paraffin, and stained with hematoxylin and eosin to assess steatosis, inflammation, and necrosis. Liver pathology was scored in a blinded manner by an outside expert as follows:

1. Degree of steatosis, score 0-4 (based on % of hepatocytes containing lipid)

0 none

1 minimal (0-24%)

2 mild (25-49%)

3 moderate (50-74%)

4 severe (>=75%)

2. Degree of inflammatory cell infiltration, score 0-4

0 none, 1 minimal, 2 mild, 3 moderate, 4 severe

3. Degree of degeneration and necrosis, score 0-4

0 none

1 hepatocellular degeneration with no necrosis

2 minimal to mild hepatocellular necrosis (with or without accompanying degeneration)

3 moderate hepatocellular necrosis (with or without accompanying degeneration)

4 severe necrosis (with or without accompanying degeneration)

The points for each grade of histological evaluation were compiled into a total score for each liver.

Results

The results of the study on the effects of histamine dihydrochloride on the protection against early alcohol induced liver injury in a rat model are summarized below in Table 1.

TABLE 1
Histologic Findings in Liver, Mean Group Severity Scores

| Total | | | Low | High |
|---|---|---|---|---|
| Overall Score | Control | Ethanol | Histamine | Histamine |
| Week 2 | 2.67 | 2.33 | 3.33 | 2.00 |
| Week 4 | 2.63 | 3.86 | 3.17 | 2.57 |

Severity scores for each finding in each group were summed and then divided by the number of animals evaluated per group. The groups were assigned scores as follows: None=0; Minimal=3; Mild=6; Moderate=9; Severe=12. Using this method, the trend between groups is demonstrated. A score for an untreated rat on normal food would be 1.00.

Animals fed a high-fat liquid diet and ethanol developed a typical histology in the liver of alcoholic liver injury, including steatosis, inflammation, necrosis and increased numbers of infiltrating leukocytes mainly neutrophiles and mononuclear cells. Serum levels of alanine and aspartate transaminase (ALT/AST) were elevated approximately two to four-fold in the ethanol treated animals.

Animals treated with histamine (0.5 mg/kg or 5 mg/kg) administered twice daily by subcutaneous injection blunted liver injury in a dose dependent manner with the highest dose being most effective. This was assessed by normalization of pathology scores and serum transaminase levels. In addition, livers excised from histamine treated animals were comparative in appearance to livers from the non-ethanol treated group. The livers from ethanol treated animals were generally more pale and with greater accentuated lobular patterns.

Histamine treated animals had normal liver appearance and tolerated ethanol dosing easier. Initially, all the animals receiving ethanol lost body weight compared to the animals on the control diet. However, both histamine treated groups recovered their body weights more quickly than in the ethanol alone treated group. In addition, these groups had similar weights to the non-ethanol treated control group by the end of the study.

Conclusions

Preliminary results illustrated a trend for somewhat normal ALT activity in the control and histamine treated groups, with the ethanol control group showing a 2-3 fold increase in ALT activity. These results show that histamine significantly lessens the damage in the liver caused by chronic ethanol administration while on a high fat diet. Moreover, the results of this study suggest that histamine and histamine receptor agonists protect early alcohol-induced liver injury in rats.

### Example 2

### In Vitro Study of Hepatic NK Cell and T Cell Activity

This example illustrates the effect of histamine on the lymphocytes isolated from human liver. Three types of human lymphocytes, CD3+T-cells, CD3-/56+ NK-cells and CD3+/56+ NK/T-cells were studied regarding their roles in oxidatively induced apoptosis in vitro. All three cell types became apoptotic after incubation with autologous monocytes (MO) or granulocytes (GR) or after treatment with hydrogen peroxide, a reactive species of oxygen. Thus, at a lymphocyte to MO ratio of 1:1, 35±5% of T-cells, 55±5% of NK-cells, and 76±7% of NK/T-cells became apoptotic. Corresponding frequencies of apoptosis at a lymphocyte to GR ratio of 1:1 were 21±4% (T-cells), 30±7% (NK-cells), and 66±8% (NK/T-cells). All data are the mean±s.e.m. (n=15-30 blood donors). Apoptosis in all cell types was significantly prevented by histamine (p<0.001). The higher sensitivity to oxidatively induced apoptosis in NK/T cells and NK-cells was confirmed in experiments in which apoptosis was induced by exogenous hydrogen peroxide; at 25 μM of hydrogen peroxide, 35±5% of T-cells, 65±7% of NK-cells, and 78±7% of NK/T-cells (n=8) became apoptotic. We conclude that liver-type lymphocytes, in particular NK/T-cells, are unusually sensitive to oxidative stress. Thus, anti-oxidative agents such as histamine may be more effective in liver neoplasia or chronic infection as the result of a higher sensitivity to oxidatively induced apoptosis in liver-infiltrating lymphocytes.

### Example 3

### Co-administration of Histamine and Hepatotoxic Drug as Single Formulation

Drug-induced liver disease represents an important challenge for health care providers, the pharmaceutical industry, and regulatory bodies. Drug hepatotoxicity is the single leading cause of acute liver failure in a survey of major medical centers in the U.S. Kaplowitz, N., Hepatology 33(1): 308-310 (2001).

The majority of adverse drug events are acute and present as cytotoxic-hepatitis-like illness or cholestatic disease. Id. Other reactions to hepatotoxic drugs include chronic hepatitis, vanishing duct disease, fatty liver, non-alcoholic steatohepatitis, fibrosis, cirrhosis, granulomatous disease, veno-occlusive disease, peliosis hepatis, and benign and malignant neoplasia. Damage to the liver can occur due to the dose of drug consumed, apparent hypersensitivity reactions, or metabolic idiosyncratic reactions.

Without being limited to a particular theory, it is believed that trauma to hepatic cells in response to insults by hepatotoxic drugs may be caused or exacerbated by ROS. As discussed above, ROS can have direct effects on various cells within the liver parenchema leading to apoptosis. Another possible mechanism by which these molecules can damage hepatic cells and tissue may be related to the effect ROS have on actuator cells of the immune system. ROS production and release inhibiting compounds reduce and/or prevent the ROS mediated damage of hepatic cells and tissue caused by exposure to hepatotoxic drugs.

An individual presenting with tuberculosis is prescribed an oral tablet comprising isoniazid and an effective dose of histamine for a period of six to twelve months. During the treatment period, the patient is monitored by a physician and lab tests to check the status of the patient's liver in response to the medication are performed. Laboratory results confirm no damage to the patient's liver in the patient taking a composition comprising both isoniazid and an effective dose of histamine as compared to patients taking isoniazid alone, which may cause severe and sometimes fatal liver damage.

### Example 4

### Co-administration of Histamine Dihydrochloride and a Hepatotoxic Drug

An epileptic taking phenytoin three times a day to control seizures is supplemented with a daily oral dose of 10 mg of histamine dihydrochloride. Prior to supplementation with histamine dihydrochloride, the epileptic experiences liver damage as a consequence of taking phenytoin as confirmed by liver biopsy. After supplementation with histamine dihydrochloride, further damage to the tissue of the liver is prevented.

### Example 5

### Herbal Preparation Comprising a ROS Scavenger

Herbal remedies, vitamins and herbal supplements have been gaining in popularity over the past decade. Comfrey has been used for centuries with success in wound healing and promoting bone health. Comfrey has also been hailed as a powerful remedy for coughs, catarrh, ulcerated bowels and stomach.

The common herb comfrey can be administered as a poultice or tea drink to as an expectorant, demulcent, and/or tonic. However, consumption of comfrey has been linked to liver damage. In order to minimize the hepatotoxic effect of comfrey, an individual consuming comfrey as an herbal remedy is supplemented with 8 mg/day of catalase. No hepatic injury is observed.

### Example 6

### Prevention of Hepatic Tissue Damage Secondary to Pancreatitis

The invention contemplates the treatment of pancreatitis and other inflammatory diseases in addition to hepatitis. Inflammation caused by TNF-α and interleukins as part of an individual's immune response can also drive damage to tissue. Histamine and histamine-related compounds block these pro-inflammatory cytokines, thereby ameliorating some of the damage to cells caused by inflammation. For a detailed discussion of the role of histamine and histamine-related compounds on mitigating damage to tissue caused by pro-inflammatory cytokines, see U.S. Pat. No. 6,242,473 and U.S. patent application Ser. No. 09/139,281, hereby incorporated by reference in their entirety.

A patient presenting with acute pancreatitis is identified. The patient is administered 100 μg/kg histamine diphosphate per day over a period of one to two weeks. A liver biopsy is performed upon resolution of the pancreatitis. The administration of histamine diphosphate prevents damage to hepatic tissue and lowers the incidence of infection associated with pancreatitis.

### Example 7

### Treatment of Bacterial Infection of the Liver

An individual presenting with a bacterial infection caused by a species of Helicobacter is identified. The individual is administered antibiotics in concert with an effective dose of a histamine prodrug. The period of infection is reduced and the trauma to hepatic cells is minimized.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A method for inhibiting and reducing reactive oxygen species (ROS)-mediated oxidative damage to hepatic cells and tissues of a subject comprising the step of:
administering a compound effective to reduce the amount of ROS in an individual suffering from a liver disease caused or exacerbated by ROS-mediated oxidative damage.

2. The method of claim 1, wherein the ROS is released constitutively.

3. The method of claim 1, wherein said liver disease is selected from the group consisting of Portal hypertension, Alagille Syndrome, Alpha-1-Antitrypsin Deficiency, Autoimmune Hepatitis, Biliary Atresia, Chronic Hepatitis, Cancer of the Liver, Cancer metastatic to the liver, Cirrhosis, Intrahepatic cholestasis, Hepatic Vein Thrombosis, Hepatic Veno-Occlusive Disease, Hepatolenticular Degeneration, Hepatomegaly, Hepatopulmonary Syndrome, Hepatorenal Syndrome, Liver Cysts, Liver Abscesses, Fatty Liver, Galactosemia, Gilbert's Syndrome, Portal Hypertension, Alcoholic Liver Disease (ALD), Parasitic Liver Diseases, Peliosis Hepatis, Erythrohepatic Porphyria, Hepatic Porphyria, Hepatic Tuberculosis, Primary Biliary Cirrhosis, Primary Sclerosing Cholangitis, Reye's Syndrome, Sarcoidosis, Tyrosinemia, Type I Glycogen Storage Disease, Wilson's Disease, Neonatal Hepatitis, NonAlchoholic SteatoHepatitis, Hemochromatosis, and Zellweger Syndrome.

4. The method of claim 1, wherein the compound effective to reduce the amount of ROS is an individual is selected from the group consisting of a compound effective to inhibit the production or release of enzymatically produced ROS, an ROS scavenger, and combinations thereof.

5. The method of claim 4, wherein said compound effective to inhibit the production or release of enzymatically produced ROS is selected from the group consisting of histamine, histamine receptor agonists, NADPH oxidase inhibitors, serotonin and serotonin agonists.

6. The method of claim 4, wherein the administration of the ROS scavenger results in ROS scavenger catalyzed decomposition of ROS.

7. The method of claim 4, wherein the scavenger is selected from the group consisting of catalase, glutathione peroxidase, ascorbate peroxidase, superoxide dismutase, glutathione peroxidase, ascorbate peroxidase, vitamin A, vitamin E, and vitamin C.

8. The method of claim 1, wherein said liver disease is selected from the group consisting of bacterial infection, fungal infection, and protozoan infection.

9. The method of claim 8, further comprising administering an antibiotic.

10. The method of claim 9, wherein said antibiotic is administered substantially simultaneously with said compound effective to inhibit the production or release of enzymatically produced ROS.

11. The method of claim 9, wherein said antibiotic is administered within 24 hours of said compound effective to inhibit the production or release of enzymatically produced ROS.

12. A method for treating a subject suffering from a disease state wherein phagocyte produced, reactive oxygen species (ROS)-mediated oxidative damage can occur, comprising the steps of:
identifying a subject with a liver disease in which ROS cause ROS-meditated oxidative damage; and
administering a compound effective to reduce the amount of ROS in said individual.

13. The method of claim 12, wherein said wherein said liver disease is selected from the group consisting of Portal hypertension, Alagille Syndrome, Alpha-1-Antitrypsin Deficiency, Autoimmune Hepatitis, Biliary Atresia, Chronic Hepatitis, Cancer of the Liver, Cancer metastatic to the liver, Cirrhosis, Intrahepatic cholestasis, Hepatic Vein Thrombosis, Hepatic Veno-Occlusive Disease, Hepatolenticular Degeneration, Hepatomegaly, Hepatopulmonary Syndrome, Hepatorenal Syndrome, Liver Cysts, Liver Abscesses, Fatty Liver, Galactosemia, Gilbert's Syndrome, Portal Hypertension, Alcoholic Liver Disease (ALD), Parasitic Liver Diseases, Peliosis Hepatis, Erythrohepatic Porphyria, Hepatic Porphyria, Hepatic Tuberculosis, Primary Biliary Cirrhosis, Primary Sclerosing Cholangitis, Reye's Syndrome, Sarcoidosis, Tyrosinemia, Type I Glycogen Storage Disease, Wilson's Disease, Neonatal Hepatitis, NonAlchoholic SteatoHepatitis, Hemochromatosis, and Zellweger Syndrome.

14. The method of claim 12, wherein said compound is selected from the group consisting of a compound effective to inhibit the production or release of enzymatically produced ROS, an ROS scavenger, and combinations thereof.

15. The method of claim 14, wherein said compound effective to inhibit the production or release of enzymatically produced ROS is selected from the group consisting of histamine, histamine receptor agonists, serotonin, serotonin agonists, and NADPH oxidase inhibitors.

16. The method of claim 14, wherein the administration of said ROS scavenger results in the reactive oxygen metabolites scavenger catalyzed decomposition of reactive oxygen metabolites.

17. The method of claim 14, wherein said reactive oxygen metabolites scavenger is selected from the group consisting of catalase, superoxide dismutase, glutathione peroxidase, and ascorbate peroxidase.

18. A composition comprising a compound effective to reduce the amount of ROS in an individual and a hepatotoxic drug.

19. The composition of claim 18, wherein said hepatotoxic drug is selected from the group consisting of azathioprine, methyldopa, nitrofuratoin, clofibrate, troglizatzone, ibuprofen, allopurinol, indomethacin, leflunomide, acetaminophen, diclofenac, isoniazid, tetracycline, erythromycin, nitrofuratoin, amoxicillin, rifampin, ketoconazole, flucloxacillin, trovafloxacin, sulfonamides, estradiol, iron, glutathione, halothane, isoflurane, captopril, diltiazem, phenytoin, valproic acid, cabamazepine, phenobarbitone, primidone, trazodone, chlorpromazine, quinidine, procainamide, amiodarone, methotrexate, cyclophosphamide, corticosteroid, anabolic steroid, glucocorticoids, pyrazinamide, para-amino salicylic acid, ethionamide, trimethoprim-sulfamethoxazole, pentamidine, zidovudine, dideoxyinosine, penicillins, and cephalosporins.

20. The composition of claim 18, wherein said hepatotoxic drug is a herbal preparation selected from the group consisting of chapparal (Larrea tridentata); germander (Teucrium chamaedrys); jin bu huan; ma huang; valerian root (Valeriana officinalis); skullcap (Scutellaria galericulata); mistletoe (Viscum species); Jamaican bush tea; senna (Cassia angustfolia); comfrey (Symphytum officinale); and kava root extract.

21. The composition of claim 18, wherein said compound is selected from the group consisting of a compound effective to inhibit the production or release of enzymatically produced ROM, a ROM scavenger, and combinations thereof.

22. The composition of claim 21, wherein said compound effective to inhibit the production or release of enzymatically produced ROM is selected from the group consisting of histamine, histamine receptor agonists, NADPH oxidase inhibitors, serotonin and serotonin agonists.

23. The composition of claim 21, wherein said ROS scavenger is selected from the group consisting of catalase, superoxide dismutase, glutathione peroxidase, and ascorbate peroxidase.

24. A method of reducing the hepatotoxicity of a drug comprising:
administering to an individual taking a hepatotoxic drug an effective dose of a compound effective to reduce the amount of ROS in an individual.

25. The method of claim 24, wherein said compound effective to reduce the amount of ROS in an individual is selected from the group consisting of a compound effective to inhibit the production or release of ROS, a ROS scavenger, and combinations thereof.

26. The method of claim 25, wherein said compound effective to inhibit the production or release of ROS is selected from the group consisting of histamine, histamine receptor agonists, NADPH oxidase inhibitors, serotonin and serotonin agonists.

27. The method of claim 25, wherein the step of administering said ROS scavenger results in ROS scavenger catalyzed decomposition of ROS.

28. The method of claim 25, wherein the scavenger is selected from the group consisting of catalase, glutathione peroxidase, ascorbate peroxidase, superoxide dismutase, glutathione peroxidase, ascorbate peroxidase, vitamin A, vitamin E, and vitamin C.

29. The method of claim 24, wherein said hepatotoxic drug is selected from the group consisting of azathioprine, methyldopa, nitrofuratoin, clofibrate, and troglizatzone.

30. The method of claim 24, wherein said hepatotoxic drug is an anti-arthritic drugs selected from the group consisting of ibuprofen, allopurinol, indomethacin, leflunomide, acetaminophen, and diclofenac.

31. The method of claim 24, wherein said hepatotoxic drug is an antibiotic selected from the group consisting of isoniazid, tetracycline, erythromycin, nitrofuratoin, amoxicillin, rifampin, ketoconazole, flucloxacillin, trovafloxacin, and sulfonamides.

32. The method of claim 24, wherein said hepatotoxic drug is selected from the group consisting of estradiol, iron, and glutathione.

33. The method of claim 24, wherein said hepatotoxic drug is selected from the group consisting of halothane and isoflurane.

34. The method of claim 24, wherein said hepatotoxic drug is selected from the group consisting of captopril, diltiazem, phenytoin, valproic acid, cabamazepine, phenobarbitone, primidone, trazodone, chlorpromazine; quinidine, procainamide, and amiodarone.

35. The method of claim 24, wherein said hepatotoxic drug is a chemotherapeutic agent.

36. The method of claim 24, wherein said hepatotoxic drug is selected from the group consisting of corticosteroid, anabolic steroids, and glucocorticoids.

37. The method of claim 24, wherein said hepatotoxic drug is used to treat HIV/AIDS patients.

38. The method of claim 37, wherein said drug is selected from the group consisting of pyrazinamide, para-amino salicylic acid, ethionamide, trimethoprim-sulfamethoxazole, pentamidine, zidovudine, dideoxyinosine, penicillins, and cephalosporins.

39. The method of claim 24, wherein said drug is a herbal preparation selected from the group consisting of chapparal (Larrea tridentata); germander (Teucrium chamaedrys); jin bu huan; ma huang; valerian root (Valeriana officinalis); skullcap (Scutellaria galericulata); mistletoe (Viscum species); Jamaican bush tea; senna (Cassia angustfolia); comfrey (Symphytum officinale); and kava root extract.

40. A method of reducing hepatic tissue damage associated with exposure to an environmental or industrial toxin comprising:
administering to a subject in need thereof an effective amount of a compound effective to reduce the amount of ROS in an individual.

41. The method of claim 40, wherein said compound is selected from the group consisting of a compound effective to inhibit the production or release of ROS, a ROS scavenger, and combinations thereof.

42. The method of claim 41, wherein said compound effective to inhibit the production or release of ROS is selected from the group consisting of histamine, histamine receptor agonists, NADPH oxidase inhibitors, serotonin and serotonin agonists.

43. The method of claim 41, wherein the step of administering said ROS scavenger results in ROS scavenger catalyzed decomposition of ROS.

44. The method of claim 41, wherein the scavenger is selected from the group consisting of catalase, glutathione peroxidase, ascorbate peroxidase, superoxide dismutase, glutathione peroxidase, ascorbate peroxidase, vitamin A, vitamin E, and vitamin C.

45. The method of claim 40, wherein said toxin is a genus of poisonous mushroom selected from the group consisting of Amanita, Galerina, and Gyromitra.

46. The method of claim 40, wherein said toxin is selected from the group consisting of cigarette smoke, diethanoloamine, sodium laurel sulfate, propylene glycol, pesticides, food additives, food preservatives, heavy metals, formaldehyde, bromobenzene, and chlorinated solvents.

47. The method of claim 46, wherein said chlorinated solvent is selected from the group consisting of dioxins, flurans, TCE, PCE, DCE, tetrachloroethylene, carbon tetrachloride, and vinyl chloride.
